(19) United States
(12) Patent Application Publication
(10) Pub. No.: US 2020/0333328 A1
(43) Pub. Date: Oct. 22, 2020

(54) APPARATUS AND METHOD FOR SIMULATING BIOLOGICAL CONDITION USING ROTATIONAL FORCE

(71) Applicants: SNU R&DB Foundation, Seoul (KR); Seoul National University Hospital, Seoul (KR)

(72) Inventors: Seung Hee YANG, Seoul (KR); Jin Woo CHOI, Seoul (KR); Dong Ki KIM, Seoul (KR); Jung Pyo LEE, Seoul (KR); Yon Su KIM, Seoul (KR); Young Chul KIM, Seoul (KR); Hee Chan KIM, Seoul (KR); Mi Yeon YU, Seoul (KR); Sa Ram LEE, Seoul (KR)

(21) Appl. No.: 16/921,419

(22) Filed: Jul. 6, 2020

Related U.S. Application Data
(62) Division of application No. 15/915,659, filed on Mar. 8, 2018.

(30) Foreign Application Priority Data
Mar. 10, 2017 (KR) 10-2017-0030826
Feb. 5, 2018 (KR) 10-2018-0014250

(51) Int. Cl. G01N 33/50; C12M 1/42; C12M 1/34; C12M 3/04

(52) U.S. Cl. CPC G01N 33/5091; C12M 27/10; C12M 41/40; C12M 35/04

(57) ABSTRACT

A biological environment simulating apparatus using rotational force includes a mounting unit on which a cell to be pressurized is mounted, a rotational force application unit configured to apply centripetal force to the mounting unit to make the mounting unit perform a circular movement along a circular path about a predetermined center point and a control unit. The control unit controls the rotational force application unit based on a type of the cell and an appropriate pressure condition matched with the type of the cell so that a pressure satisfying the appropriate pressure condition is applied to the cell.

### CROSS-REFERENCES TO RELATED APPLICATIONS

This application is a divisional application of U.S. patent application Ser. No. 15/915,659 filed on Mar. 8, 2018, which claims priority to and benefit of from Korean Patent Applications No. 10-2017-0030826 filed on Mar. 10, 2017 and No. 10-2018-0014250 filed on Feb. 5, 2018, the disclosures of which are incorporated herein in their entirety by reference.

### FIELD OF THE INVENTION

The disclosure relates to an apparatus and a method for simulating a biological environment for a test target cell by applying a pressure obtained by rotation to the test target cell.

### BACKGROUND OF THE INVENTION

Hypertension is a chronic disease in which the blood pressure is higher than a normal range. Generally, adults over the age of 18 with a systolic blood pressure higher than 140 mmHg or a diastolic blood pressure higher than 90 mmHg are diagnosed with hypertension.

In most patients with hypertension, there was no symptom. However, hypertension may cause complications such as stroke, heart failure, retinopathy, coronary artery disease, renal failure, and peripheral vascular disease. Recently, it has become known that kidney injury due to high blood pressure may result in a decreased glomerular function and extensive fibrosis of the kidney tissue.

Cells in patients with hypertension are subjected to a higher pressure compared to normal cells. Therefore, in order to study cells in a high blood pressure condition, it is required to simulate a biological environment similar to that of the patient with high blood pressure by forcibly applying pressure to the cells.

Generally, a method for chemically damaging cells for the above-mentioned simulation is employed. However, such a method is disadvantageous in that the cells are damaged by the drugs used. Recently, a method for simulating the high blood pressure condition by applying force to cells directly is employed additionally. However, this method has disadvantages in that it has low reproducibility and needs an apparatus with a complicated structure and a lot of space. Therefore, a considerably high cost is required for practical use thereof.

Further, when pressure is applied without consideration of the type of cell, it is difficult to accurately simulate the hypertensive environment.

### SUMMARY OF THE INVENTION

In view of the above, the disclosure provides an apparatus and method capable of stably applying pressure to a cell with consideration of the type of the cell and the blood pressure of the body to be simulated.

However, the object of the present disclosure is not limited to the above-described object and may include other objects that can be clearly understood by those skilled in the art to which the present disclosure pertains from the following description.

In accordance with an aspect of the present disclosure, there is provided a biological environment simulating apparatus using rotational force. The apparatus includes a mounting unit on which a cell to be pressurized is mounted, a rotational force application unit configured to apply centripetal force to the mounting unit to make the mounting unit perform a circular movement along a circular path about a predetermined center point and a control unit configured to control the rotational force application unit based on a type of the cell and an appropriate pressure condition matched with the type of the cell so that a pressure satisfying the appropriate pressure condition is applied to the cell.

Further, the apparatus may include an input unit configured to receive the type of the cell from a user of the apparatus and a database in which the appropriate pressure condition matched with the type of the cell is stored.

Further, the input unit may be configured to receive a blood pressure set value from the user, the appropriate pressure condition may include information on a relation between a blood pressure of a body and a pressure applied to the cell on an assumption that the cell exists in the body, and the control unit, based on the information included in the appropriate pressure condition and under an assumption that the cell exists in a body whose blood pressure matches the blood pressure set value, may be configured to calculate a value of an actual pressure expected to be applied to the cell and control the rotational force application unit so that a pressure matching the value of the actual pressure is applied to the cell.

Further, the control unit may be configured to calculate a speed of the circular movement of the mounting unit that satisfies the appropriate pressure condition and control the rotational force application unit to rotate the mounting unit at the calculated speed.

Further, the rotational force application unit may include a rotation shaft extending through the center point and a support arm extending radially from the rotation shaft and supporting the mounting unit, and the mounting unit may include a space where a container containing the cell is accommodated.

Further, the appropriate pressure condition may include information on a pressure range in which the cell is able to survive for a predetermined critical time with a predetermined critical probability.

In accordance with another aspect of the present disclosure, there is provided a biological environment simulating method performed by the biological environment simulating apparatus. The method includes steps of calculating, based on a radius of rotation of a mounting unit of the apparatus, a type of a cell mounted on the mounting unit and an appropriate pressure condition matched with the type of the cell, a speed of a circular movement for applying a pressure satisfying the appropriate pressure condition to the cell and controlling the mounting unit to perform the circular movement at the calculated speed along a circular path having a radius matching the radius of rotation by applying centripetal force to the mounting unit.

Further, the method may include steps of receiving a blood pressure set value from a user of the apparatus, the appropriate pressure condition may include information on a relation between a blood pressure of a body and a pressure applied to the cell on an assumption that the cell exists in the body, and said calculating the speed of the circular movement may include steps of calculating, based on the information included in the appropriate pressure condition and under an assumption that the cell exists in a body whose blood pressure matches the blood pressure set value, a value of an actual pressure expected to be applied to the cell and calculating the speed of the circular movement for applying a pressure corresponding to the value of the actual pressure to the cell.

Further, the appropriate pressure condition may include information on a pressure range in which the cell is able to survive for a predetermined critical time with a predetermined critical probability.

In accordance with an embodiment of the present disclosure, a desired pressure can be applied to a test target cell by using the centrifugal force generated by the rotation. By using the centrifugal force, the pressure can be stably applied to the cell without damaging the cell. In addition, easy manipulation and highly repetitive reproducibility can be ensured. Further, by setting an appropriate pressure condition for each type of test target cell, a realistic hypertension environment can be simulated.

In accordance with an embodiment of the present disclosure, it is possible to study the operations and reactions of various cells in a patient's body under various blood pressures of the patient. The embodiment of the present disclosure can be utilized for the development of drugs for treating hypertension and analysis of the efficacy of drugs, and ultimately contribute to the improvement of the health and the quality of life of hypertension patients.

### BRIEF DESCRIPTION OF THE DRAWINGS

The objects and features of the disclosure will become apparent from the following description of embodiments, given in conjunction with the accompanying drawings, in which:

FIGS. 1 and 2 explain a configuration of an apparatus for simulating a biological environment according to an embodiment;

FIG. 3 explains the steps of a method for simulating a biological environment according to an embodiment that uses the apparatus for simulating a biological environment; and

FIGS. 4A to 4C and 5A to 5B show the results of tests using the apparatus for simulating a biological environment according to the embodiment.

### DETAILED DESCRIPTION OF THE EMBODIMENTS

The advantages and features of the embodiments and the methods of accomplishing the embodiments will be clearly understood from the following description taken in conjunction with the accompanying drawings. However, embodiments are not limited to those embodiments described, as embodiments may be implemented in various forms. It should be noted that the present embodiments are provided to make a full disclosure and also to allow those skilled in the art to know the full range of the embodiments. Therefore, the embodiments are to be defined only by the scope of the appended claims.

In describing the embodiments of the present disclosure, if it is determined that the detailed description of related known components or functions unnecessarily obscures the gist of the present disclosure, the detailed description thereof will be omitted. Further, the terminologies to be described below are defined in consideration of the functions of the embodiments of the present disclosure and may vary depending on a user's or an operator's intention or practice. Accordingly, the definition thereof may be made on a basis of the content throughout the specification.

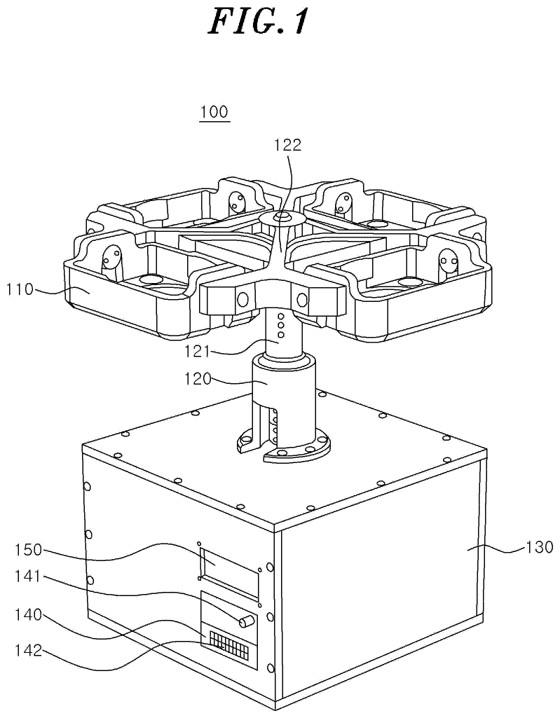

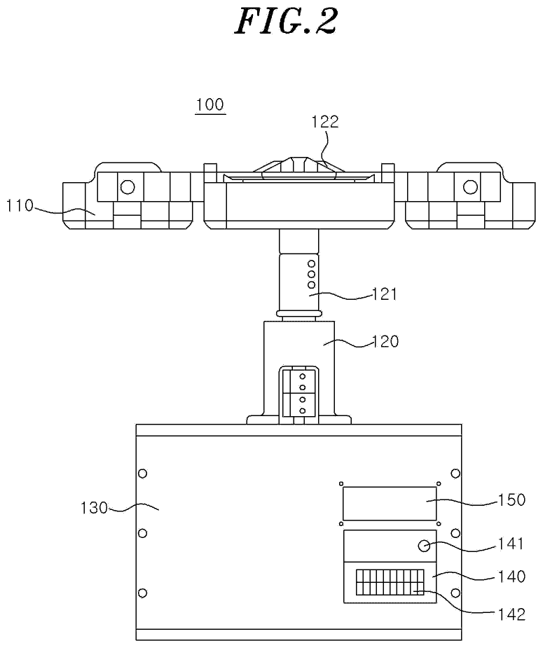

FIGS. 1 and 2 explain a configuration of an apparatus for simulating a biological environment according to an embodiment. Specifically. FIG. 1 is a perspective view of the biological environment simulating apparatus 100, and FIG. 2 is a front view of the biological environment simulating apparatus 100. The biological environment simulating apparatus 100 may include a mounting unit 110, a rotational force application unit 120, a control unit 130, an input unit 140, and an output unit 150. However, the biological environment simulating apparatus 100 shown in FIGS. 1 and 2 is only one embodiment of the present disclosure, and the idea of the present disclosure is not limited by FIGS. 1 and 2.

The biological environment simulating apparatus 100 can simulate a hypertension environment by circularly moving a test target cell mounted on the mounting unit 110 to apply a centrifugal force resulting from the circular movement to the test target cell.

The cell pressurized by the centrifugal force becomes similar to a cell pressurized in a hypertension patient's body. In accordance with the disclosure, the magnitude of the pressure applied to the cell can be appropriately controlled by controlling the speed of the circular movement.

Such a method can be relatively simply implemented as described above and allows for the stable simulation of a hypertension environment without damaging the cells compared to a method that applies physical or chemical changes.

As can be seen from FIGS. 1 and 2, the mounting unit 110 may have a space where a container containing cells can be accommodated. An inner space of the container may be set to an environment suitable for culturing cells. Since it is unnecessary to take the cells out of the container due to the structure of the mounting unit 110, it is possible to continuously maintain the environment suitable for the cells during the operation of the biological environment simulating apparatus 100.

The rotational force application unit 120 applies centripetal force to the mounting unit 110 so that the mounting unit 110 can perform a circular movement along a circular path about a predetermined center point. To that end, the rotational force application unit 120 may include a power unit such as a general motor or the like. The rotational force application unit 120 may further include a rotation shaft 121 vertically extending through the center point of the circular path and a support arm 122 extending radially from the rotation shaft.

Referring to FIG. 1, the mounting unit 110 is connected to the rotation shaft 121 through the support arm 122. More specifically, the support arm 122 extends radially from the rotation shaft 121 and the mounting unit 110 is supported by the support arm 122. Accordingly, the mounting unit 110 can move circularly at an angular velocity that is the same as a rotational angular velocity of the rotation shaft 121. The radius of rotation of the mounting unit 110 is determined by the horizontal distance between the center of the mounting unit 110 and the rotation shaft 121.

The control unit 130 can control the pressure applied to the cell in the mounting unit 110 by controlling the rotating speed of the rotation shaft 121. More specifically. based on the type of the cell and an appropriate pressure condition matched with the type of the cell, the control unit 130 can apply a pressure satisfying the appropriate pressure condition to the cell. In other words, the control unit 130 can calculate the speed of the circular movement of the mounting unit 110 that satisfies the appropriate pressure condition and control the rotational force application unit 120 to rotate the mounting unit at the calculated speed.

The control unit 130 may include an arithmetic unit such as a microprocessor or the like. In that case, the control unit 130 may include a box-shaped main body having therein the arithmetic unit and provide support to the rotational force application unit 120. However, the control unit 130 is not necessarily limited thereto.

For the operation of the control unit 130, the information on the type of the test target cell or the like can be inputted into the input unit 140 by a user of the biological environment simulating apparatus 100.

The biological environment simulating apparatus 100 may further include a database (not shown). The database may contain the information required for the operation of the biological environment simulating apparatus 100, such as the appropriate pressure condition matched with each cell type, i.e., the appropriate pressure condition determined for each cell type, or the like.

The output unit 150 can provide the information required for the operation of the biological environment simulating apparatus 100 (e.g., the rotation speed of the mounting unit 110 or the like) to the user.

From the viewpoint of hardware, the input unit 140 and the output unit 150 may be provided outside the main body of the control unit 130 and the database may be provided inside the main body of the control unit 130. The input unit 140 may include a rotary switch 141 for controlling the rotation of the rotation shaft 121 that causes the circular movement of the mounting unit 110 or a keypad 142 for receiving the input of more specific information from the user (e.g., a blood pressure set value to be described later, an appropriate pressure condition to be stored in the database, or the like). The output unit 150 may include a visual output device such as a display or the like, or an auditory output device such as a speaker or the like.

The database can be implemented by a computer-readable storage medium. The computer-readable storage medium may be, e.g., magnetic media such as a hard disk, a floppy disk, a magnetic tape, optical media such as a CD-ROM or a DVD, magneto-optical media such as a floptical disk, or a hardware device such as flash memory configured to store and execute program instructions.

As described above, in accordance with one embodiment of the present disclosure, it is possible to easily simulate an environment similar to the inside of the body of a hypertension patient by using pressure obtained from centrifugal force. Further, in accordance with one embodiment of the present disclosure, various convenient functions can be provided to a user by using the appropriate pressure condition determined for each test target cell type. Those functions will be described hereinafter.

The appropriate pressure condition stored in the database may include various kinds of information. For example, the appropriate pressure condition can include information on a pressure range in which a test target cell can survive for a predetermined critical time with a predetermined critical probability. This is because the appropriate pressure condition in which a certain rate of survival is ensured may be different depending on the cell. Based on the appropriate pressure condition, the control unit 130 can control the pressure to be applied to the test target cell that is inputted through the input unit 140 within a range matched with the type of the test target cell.

Further, in accordance with an embodiment of the present disclosure, when the input unit 140 receives a blood pressure set value from a user, the control unit 130 can calculate the value of the actual pressure that is expected to be applied to the test target cell on the assumption that the test target cell exists in a body with blood pressure matches the blood pressure set value. Then, the control unit 130 can control the rotational force application unit 120 so that a pressure matching the value of the actual pressure is applied to the test target cell. In other words, when a user inputs the type of the test target cell and the blood pressure of a body to be simulated through the input unit 140, the control unit 130 automatically determines the speed of the circular movement of the mounting unit 110 based on the information inputted by the user and the appropriate pressure condition.

For example, in the case of testing glomerular vascular endothelial cells in a hypertension patient with systolic blood pressure of 160 mmHg, a user can mount the glomerular vascular endothelial cells on the mounting unit 110, input “160 mmHg” as the blood pressure set value and “glomerular vascular endothelial cell” as the cell type through the input unit 140, and start the operation of the biological environment simulating apparatus 100. Accordingly, the pressure applied to the glomerular vascular endothelial cells mounted on the mounting unit 110 as the test target cell becomes equal to the pressure that is actually applied to the glomerular vascular endothelial cells in the hypertension patient with a systolic blood pressure of 160 mmHg.

With this function, it is possible to more effectively simulate a situation in which different pressures may be applied even to the same body depending on the type of cell. In order to realize this function, the appropriate pressure condition may include information on the relation between the blood pressure of a body and the pressure applied to a cell under the assumption that the cell exists in the body. The information may be prepared for each cell type.

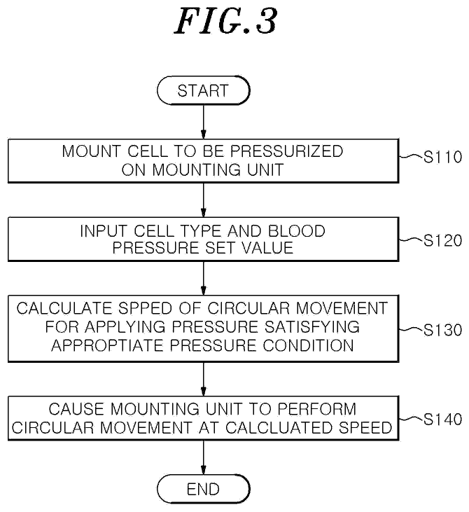

FIG. 3 explains the steps of a method for simulating a biological environment according to an embodiment of the present disclosure that uses the biological environment simulating apparatus. The steps of the method shown in FIG. 3 are not necessarily executed in that order and the sequence thereof may be changed, if necessary. Redundant description of the same parts in FIGS. 1 and 2 may be omitted.

First, a cell to be pressurized is mounted on the mounting unit 110 (S10). Next, the cell type and the blood pressure set value are inputted from a user through the input unit 140 (S120). Then, the control unit 130 calculates the speed of a circular movement of the mounting unit 110 for applying a pressure satisfying an appropriate pressure condition to the cell based on the radius of rotation of the mounting unit 110, the cell type, and the appropriate pressure condition matched with the cell type (S130). Lastly, the control unit 130 controls the rotational force application unit 120 so that the rotational force application unit 120 can cause the mounting unit 110 to perform the circular movement at the calculated speed along a circular path having a radius matching the radius of rotation by applying centripetal force to the mounting unit 110 (S140).

In step S130, the control unit 130 can calculate the value of the actual pressure expected to be applied to the cell on the assumption that the cell exists in a body whose blood pressure matches the blood pressure set value based on the appropriate pressure condition. Further, in step S130, the control unit 130 can calculate a speed of the circular movement for applying the pressure matching the value of the actual pressure to the cell.

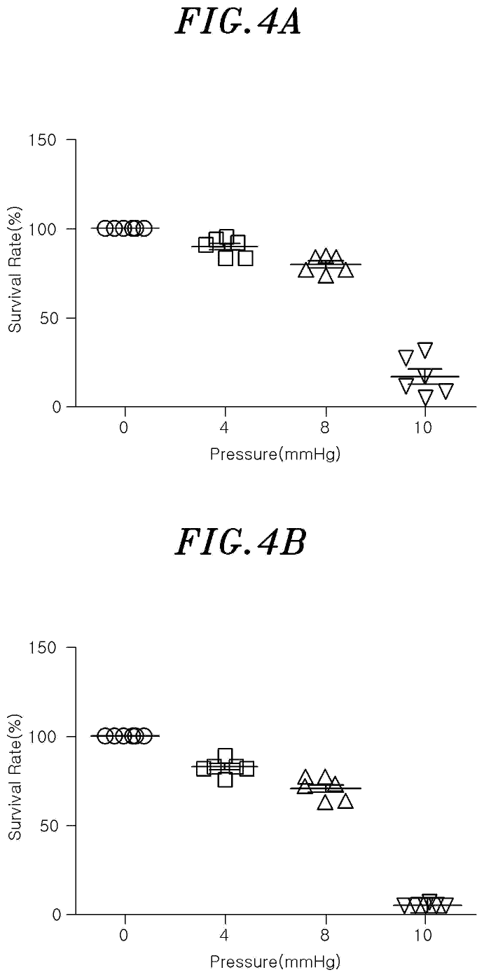

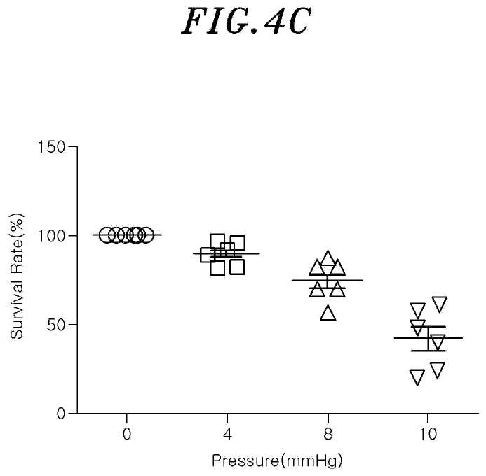

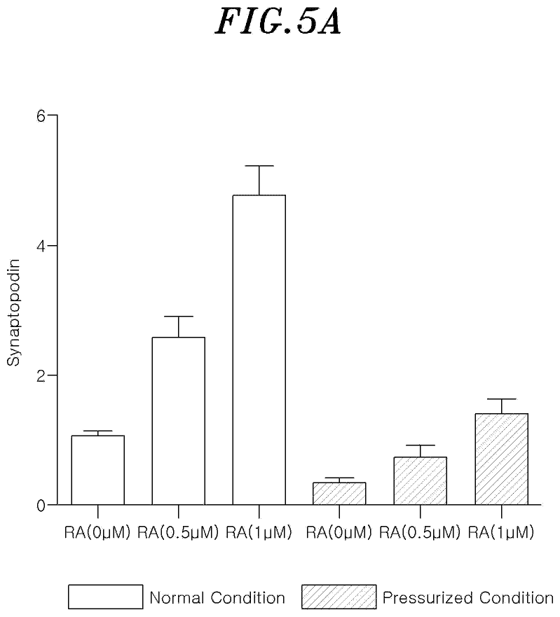

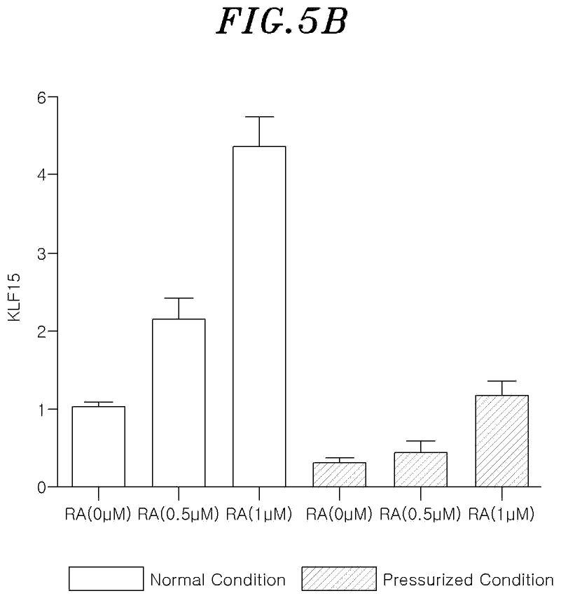

FIGS. 4A to 4C show the results of the tests using the biological environment simulating apparatus according to the embodiment of the present disclosure, and FIGS. 5A to 5B show the results of the tests after adding the retinoic acid under different pressure conditions.

FIGS. 4A to 4C show the survival rates of three types of kidney cells depending on a pressure to be applied thereto. FIGS. 4A to 4C show the results of the tests on podocytes, vascular endothelial cells, and mesangial cells, respectively. The tests were executed under four conditions: i.e., a condition in which a pressure was not applied, a condition in which a pressure of 4 mmHg was applied, a condition in which a pressure of 8 mmHg was applied, and a condition in which a pressure of 10 mmHg was applied. The cells that survived for 48 hours were considered to be alive.

When pressure was not applied, the survival rates of all the cells were close to 100%. However, as the pressure was increased, the survival rate of the vascular endothelial cells decreased relatively rapidly. The decrease in the survival rate of the podocytes was relatively slow compared to that of the vascular endothelial cells. While two other cells had survival rates of close to 0 when a pressure of 10 mmHg was applied, the survival rate of the mesangial cells was relatively higher.

As can be seen from FIGS. 4A to 4C, the relation between the pressure and the survival rate varies depending on the type of cell, and the appropriate pressure condition for each cell type can be set based on the results of the tests.

FIGS. 5A and 5B show the results of the tests on the effect of retinoic acid on the podocyte, obtained by using the biological environment simulating apparatus 100 according to the embodiment of the present disclosure. The pressure was applied to the podocyte under two conditions: i.e., a normal condition in which pressure was not applied to the podocyte and a condition in which pressure of 4 mmHg was applied. The retinoic acid was applied to the podocyte at three concentrations: 0 μM, 0.5 μM, and 1 μM. Therefore, six conditions can be obtained from the combination of the pressurized condition and the retinoic acid concentration condition.

Referring to FIG. 5A, the amount of the podocyte differentiation marker (synaptopodin) is increased as the concertation of the retinoic acid is increased. However, the amount of the differentiation marker of the podocyte is smaller under the pressurized condition than under the normal condition. Referring to FIG. 5B, the transcription factor of podocyte differentiation (KLF 15) shows the same tendency as that shown in FIG. 5A.

Referring to FIGS. 5A and 5B, retinoic acid tends to induce the differentiation of podocyte even when the pressure is applied to the podocyte in a similar manner to that in the normal state. From the above, it is clear that the efficacy of retinoic acid-containing drugs can be obtained even in an environment in which a pressure similar to that of the body exists.

The combinations of respective sequences of a flow diagram attached herein may be carried out by computer program instructions. Since the computer program instructions may be executed by processors of a general purpose computer, a special purpose computer, or other programmable data processing apparatus, the instructions, executed by the processor of the computer or other programmable data processing apparatus, create means for performing functions described in the respective sequences of the sequence diagram. The computer program instructions, in order to implement functions in a specific manner, may be stored in a memory useable or readable by the computer or a computer for other programmable data processing apparatus, and the instructions stored in the memory useable or readable by a computer may produce manufacturing items including an instruction means for performing functions described in the respective sequences of the sequence diagram. The computer program instructions may be loaded in a computer or other programmable data processing apparatus, and therefore, the instructions, which are a series of sequences executed in a computer or other programmable data processing apparatus to create processes executed by a computer to operate a computer or other programmable data processing apparatus, may provide operations for executing functions described in the respective sequences of the flow diagram.

Moreover, the respective sequences may refer to two or more modules, segments, or codes including at least one executable instruction for executing a specific logical function(s). In some alternative embodiments, it is noted that the functions described in the sequences may be run out of order. For example, two consecutive sequences may be substantially executed simultaneously or often in reverse order according to the corresponding functions.

The above description illustrates the technical idea of the present disclosure, and it will be understood by those skilled in the art to which this present disclosure belongs that various changes and modifications may be made without departing from the scope of the essential characteristics of the present disclosure. Therefore, the exemplary embodiments disclosed herein are not used to limit the technical idea of the present disclosure, but to explain the present disclosure, and the scope of the technical idea of the present disclosure is not limited by those embodiments. Therefore, the scope of protection of the present disclosure should be construed as defined in the following claims, and all technical ideas that fall within the technical idea of the present disclosure are intended to be embraced by the scope of the claims of the present disclosure.

What is claimed is:

1. A biological environment simulating method performed by a biological environment simulating apparatus, the method comprising:
receiving a type of a cell mounted on a mounting unit of the apparatus and a blood pressure set value from a user;
calculating a speed of a rotation of the mounting unit based on a radius of rotation of the mounting unit, the type of the cell and an appropriate pressure condition matched with the type of the cell, the rotation of the mounting unit resulting in applying a pressure satisfying the appropriate pressure condition to the cell;
controlling the mounting unit to rotate at the calculated speed along a circular track having a radius matching the radius of rotation by applying centripetal force to the mounting unit,
wherein the appropriate pressure condition includes information on a pressure range in which the cell is survivable more than a predetermined critical time with a predetermined critical probability,
the appropriate pressure condition further includes information on a relationship between a blood pressure of a body and a pressure applied to the cell on an assumption that the cell exists in the body, and
said calculating the speed of the rotation of the mounting unit includes:
calculating a value of an actual pressure expected to be applied to the cell based on the information included in the appropriate pressure condition and under an assumption that the cell exists in the body whose blood pressure matches the blood pressure set value; and
calculating the speed of the rotation of the mounting unit for applying a pressure corresponding to the value of the actual pressure to the cell.
